(19) United States
(12) Patent Application Publication
(10) Pub. No.: US 2017/0345114 A1
(43) Pub. Date: Nov. 30, 2017

(54) MANAGEMENT OF MEDICAL TRANSPORT FOR PATIENTS

(71) Applicant: EMS Loop, LLC, Arvada, CO (US)

(72) Inventors: Andrew Brandt, Boulder, CO (US); Daniel Patrick Richardson, Boulder, CO (US); Robert Durkin, Arvada, CO (US)

(21) Appl. No.: 15/603,051

(22) Filed: May 23, 2017

Related U.S. Application Data
(60) Provisional application No. 62/340,666, filed on May 24, 2016.

(51) Int. Cl. G06Q 50/30; G06Q 50/22; G08G 1/00; G06F 17/30

(52) U.S. Cl. CPC G06Q 50/30; G08G 1/202; G06Q 50/22; G06F 17/30241

(57) ABSTRACT

Systems and methods are provided for coordinating a fleet of vehicles that engage in medical transport. One embodiment is a system that includes a fleet control server that receives a request for medical transport of a patient, and that identifies a fleet of vehicles that provide medical transport. The system also includes crew devices at the vehicles that each report a position of a corresponding vehicle, and a reporting server. The fleet control server selects a vehicle in the fleet to perform medical transport of the patient based on the position of the vehicle in relation to other vehicles in the fleet, and determines an interested party that is distinct from the patient. The reporting server reports selection of the vehicle to the interested party, and transmits vehicle tracking data to the interested party in real time as the vehicle services the request.

### RELATED APPLICATIONS

This application claims priority to commonly owned U.S. provisional patent application No. 62/340,666, which was filed May 24, 2016, is entitled “Emergency Services Tracking System,” and is hereby incorporated by reference.

### FIELD

The disclosure relates to the field of medical transport, and in particular, to coordinating vehicular transport of patients in relation to medical treatment.

### BACKGROUND

Medical transportation is performed on a massive scale of operation across the country. For example, hundreds of thousands of instances of medical transport may occur during a single day. An instance of medical transport involves the vehicular conveyance of a patient from one location to another location in relation to treatment. This may involve taking the patient to or from a treatment location. For example, an instance of medical transport may involve conveying a patient to or from a hospital. Some instances of medical transport relate to emergencies (e.g., an emergency response to a car crash), while other instances of medical transport may be non-emergency in nature (e.g., the transport of a stable patient between buildings within a large hospital campus).

Medical transport service providers each utilize a fleet of vehicles to transport a wide variety of patients throughout the day. Incoming requests for medical transport may be received via a Computer Aided Dispatch (CAD) system (e.g., a CAD system for 911 emergency services), or may be received directly from a provider of medical services.

Presently, once a vehicle has been assigned to provide medical transport for a patient, the entity that made the request for medical transport must simply wait for the vehicle. This results in uncertainty while awaiting the vehicle. The uncertainty may increase tension, particularly for emergency-related instances of medical transport. Hence, medical transport service providers continue to seek out enhanced techniques for fleet management that increase the accuracy of, efficiency of, and satisfaction pertaining to medical transport services.

### SUMMARY

Embodiments described herein provide enhanced systems that facilitate coordination and exchange of information relating to instances of medical transport. For example, systems described herein may provide detailed vehicle tracking and support information to a third party, such as a bystander at a car crash, or a family member. The systems described herein may alternatively or additionally precisely track locations at which patients are picked up or dropped off, and utilize this information to enhance navigational information provided to a fleet of vehicles during medical transport. The systems described herein may even, in one embodiment, monitor and suggest revisions to the crew composition of individual vehicles within the fleet, based on interactions between crew members, medical personnel, and/or third parties at pick up and drop off locations, or may suggest that certain vehicle crews not be assigned to provide certain instances of medical transport.

One embodiment is a system that includes a fleet control server that receives a request for medical transport of a patient, and that identifies a fleet of vehicles that provide medical transport. The system also includes crew devices at the vehicles that each report a position of a corresponding vehicle, and a reporting server. The fleet control server selects a vehicle in the fleet to perform medical transport of the patient based on the position of the vehicle in relation to other vehicles in the fleet, and determines an interested party that is distinct from the patient. The reporting server reports selection of the vehicle to the interested party, and transmits vehicle tracking data to the interested party in real time as the vehicle services the request.

A further embodiment is a method that includes receiving a request for medical transport of a patient, identifying a fleet of vehicles that provide medical transport, determining a position of each of the vehicles in the fleet as reported by crew devices at the vehicles, and selecting a vehicle in the fleet to perform medical transport of the patient based on the position of the vehicle in relation to other vehicles in the fleet. The method further includes determining an interested party that is distinct from the patient, reporting selection of the vehicle to the interested party, and transmitting vehicle tracking data to the interested party in real time as the vehicle services the request.

A further embodiment is non-transitory computer readable medium embodying programmed instructions which, when executed by a processor, are operable for performing a method. The method includes receiving a request for medical transport of a patient, identifying a fleet of vehicles that provide medical transport, determining a position of each of the vehicles in the fleet as reported by crew devices at the vehicles, and selecting a vehicle in the fleet to perform medical transport of the patient based on the position of the vehicle in relation to other vehicles in the fleet. The method further includes determining an interested party that is distinct from the patient, reporting selection of the vehicle to the interested party, and transmitting vehicle tracking data to the interested party in real time as the vehicle services the request.

Other exemplary embodiments (e.g., methods and computer-readable media relating to the foregoing embodiments) may be described below. The features, functions, and advantages that have been discussed can be achieved independently in various embodiments or may be combined in yet other embodiments further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a block diagram of a medical transport environment in an exemplary embodiment.

FIG. 2 is a block diagram illustrating a fleet control server in an exemplary embodiment.

FIG. 3 is a flowchart illustrating a method for providing medical transport information to an interested party in an exemplary embodiment.

FIGS. 4A-4C are diagrams illustrating medical transport information presented to various parties in an exemplary embodiment.

FIG. 5 is a message diagram illustrating communications for emergency medical transport in an exemplary embodiment.

FIG. 6 is a message diagram illustrating communications for non-emergency medical transport in an exemplary embodiment.

FIG. 7 is a flowchart illustrating a method for engaging in location enhancement for medical transport in an exemplary embodiment.

FIG. 8 is a diagram illustrating a window utilized for location enhancement in an exemplary embodiment.

FIG. 9 is a flowchart illustrating a method for revising crew composition for a medical transport vehicle in an exemplary embodiment.

FIG. 10 is a diagram illustrating a display utilized for reporting satisfaction with crew for a medical transport vehicle in an exemplary embodiment.

FIG. 11 is a social graph illustrating relationships between crew members of a medical transport vehicle and medical staff in an exemplary embodiment.

FIG. 12 is a flowchart illustrating a method for selecting performing fleet management for medical transport services in an exemplary embodiment.

FIG. 13 is a diagram illustrating a window utilized for fleet management of medical transport services in an exemplary embodiment.

FIGS. 14-19 illustrate various messages exchanged within a medical transport environment in an exemplary embodiment.

FIG. 20 illustrates a processing system operable to execute a computer readable medium embodying programmed instructions to perform desired functions in an exemplary embodiment.

### DESCRIPTION

The figures and the following description illustrate specific exemplary embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

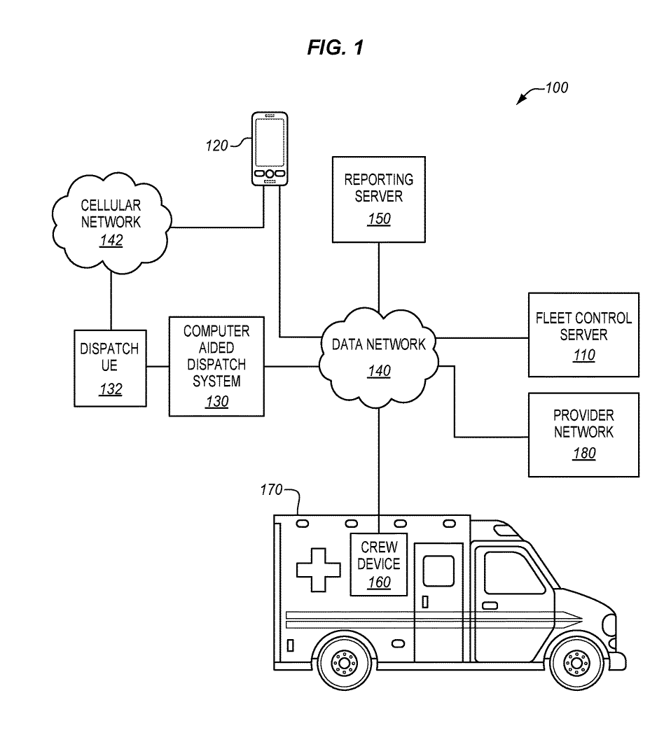

FIG. 1 is a block diagram of a medical transport environment 100 in an exemplary embodiment. Medical transport environment 100 comprises any suitable combination of vehicles, systems, and devices that interact with each other in relation to instances of medical transport for one or more patients. For example, systems within medical transport environment 100 may coordinate thousands of instances of medical transport, performed by hundreds of vehicles, throughout the day. Medical transport environment 300 has been enhanced to include a variety of components which facilitate vehicle tracking, vehicle guidance, and the exchange of information during instances of medical transport for patients.

In this embodiment, medical transport environment 100 includes mobile device 120 (e.g., a cellular phone, laptop, radio, tablet, etc.), which provides calls via cellular network 142 to dispatch User Equipment (UE) 132. Dispatch UE 132 may comprise a telephone for a dispatcher, or a component of an automated telephonic dispatch system. Based on a call received from mobile device 120, Computer Aided Dispatch (CAD) system 130 is updated with a request for medical transport. While only one CAD system 130 is illustrated in FIG. 1, in further embodiments a separate CAD system 130 may exist for each of an emergency medical services group, a fire department, a police department, etc.

CAD system 130 contacts fleet control server 110 via an Application Programming Interface (API) in order to schedule an instance of medical transport to move a patient to or from a provider of medical services/treatment. Fleet control server 110 selects vehicle 170 for performing medical transport for the patient, and may provide navigational information (e.g., Global Positioning System (GPS) coordinates for pick up and drop off locations, a route selection, etc.) to vehicle 170 via a crew device 160. Crew device 160 is an electronic device that reports progress to fleet control server 110, and may comprise a mobile device such as a cellular phone or tablet. In some embodiments, crew device 160 may even comprise a Mobile Data Terminal (MDT) at vehicle 170.

Fleet control server 110 may further receive locational and other data from crew device 160 in real-time (e.g., multiple times per second), and may provide the information to reporting server 150. Reporting server 150 provides updates to CAD system 130, mobile device 120, and/or provider network 180 (i.e., computer systems utilized by a provider of medical service/treatment) via data network 140 in order to ensure that interested parties remain informed as to the progress of vehicle 170 during medical transport.

Fleet control server 110 may also be capable of coordinating requests for medical transport that are initiated by provider network 180. For example, a server at provider network 180 may provide requests to fleet control server 110 for instances of medical transport, without interacting with CAD system 130 and/or mobile device 120.

Cellular network 142 comprises a combination of base stations and other components that operate as a Radio Access Network (RAN) in order to provide wireless telephone services, and may be coupled for communication with data network 140. Data network 140 may comprise the Internet, or another network that exchanges data. For example, data network 140 may engage in packetized communications (e.g., Internet Protocol (IP) communications) in order to exchange data. Data network 140 may comprise wired or wireless network devices, or a combination thereof.

CAD system 130 comprises any suitable device and/or system for facilitating the dispatch of vehicles for medical transport. For example, CAD system 130 may provide applications that facilitate the processing of calls from mobile device 120 (e.g., via cellular network 142). CAD system 130 may process information received via 9-1-1 emergency response for instances of emergency medical transport, and may process information for non-emergency medical transport via other systems as desired. Based on this information, CAD system 130 may generate specific requests for instances of medical transport for transmission to fleet control server 110.

Vehicle 170 comprises any suitable passenger vehicle capable of providing medical transport for patients. For example, vehicle 170 may comprise a motor vehicle such as an ambulance, may comprise an aircraft or water craft, etc. Vehicle 170 may also provide one or more types of care, such as oxygen, intravenous (IV) fluid support, Basic Life Support (BLS), Advanced Life Support (ALS), etc. Crew device 160 within vehicle 170 may comprise a secured device capable of communicating with fleet control server 110 via cellular network 142 and/or data network 140. Crew device 160 may push notifications relating to the status and location of vehicle 170 on a periodic basis (e.g., each time the status changes, every ten seconds, etc.). While only one vehicle 170 is illustrated in FIG. 1, it will be understood that any number of vehicles may be managed in real-time by fleet control server 110.

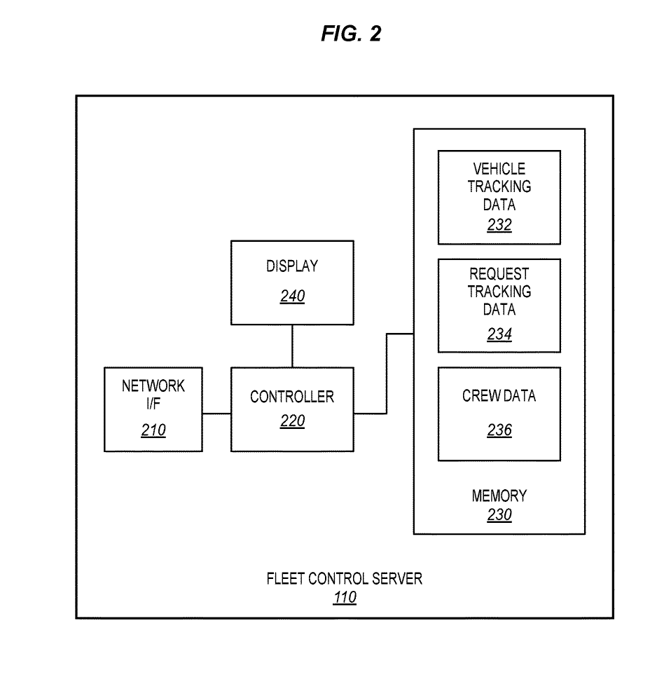

Reporting server 150 and fleet control server 110 comprise any suitable computer systems capable of managing the operations of a fleet of vehicles to provide medical transport services. For example, reporting server 150 and fleet controls server 110 may comprise hardware operating based on instructions stored in memory. While reporting server 150 is illustrated as being physically distinct from fleet control server 110, in further embodiments reporting server 150 and fleet control server 110 may be implemented on the same hardware platform. Any number of mobile devices may utilize medical transport environment 100, and each instance of medical transport may involve communications with a different mobile device. Furthermore, multiple CAD systems, dispatchers, and provider networks may interact with single fleet control server 110 as desired. Further details of fleet control server 110 are provided with respect to FIG. 2.

FIG. 2 is a block diagram illustrating a fleet control server 110 in an exemplary embodiment. In this embodiment, fleet control server 110 is described as a single computing device, although in further embodiments fleet control server 110 is implemented on multiple servers operating in parallel to provide cloud services, etc. In this embodiment, fleet control server 110 comprises network interface (I/F) 210, which receives requests for medical transport via data network 140. For example, network I/F 210 may comprise an Ethernet interface, Serial Attached Small Computer System Interface (SAS) port, wireless adapter, etc. As used herein, requests for “medical transport” include requests for medical response, even if such requests do not explicitly request the transport of a patient. Requests for medical response such as emergency 9-1-1-phone calls are expected to involve a judgment call by first responders as to whether transport of a patient is required. Hence, requests for medical response are considered to implicitly involve medical transport, even though they do not explicitly request medical transport.

Controller 220 manages the operations of fleet control server 110. For example, controller 220 may process incoming requests, select vehicle 170 from other vehicles in the fleet to provide medical transport based on an incoming request, and coordinate the exchange of information between various entities while vehicle 170 is transporting a patient. In one embodiment, controller 220 may provide a mobile and/or web application to crew device 160 that collects location, activity, and medical transport data for sharing with mobile device 120, CAD system 130, and/or provider network 180. Controller 220 may further provide a mobile and/or web application to mobile device 120 or provider network 180 that facilitates viewing of this information. Controller 220 may be implemented, for example, as custom circuitry, as a hardware processor executing programmed instructions, or some combination thereof.

Fleet control server 110 further comprises memory 230, which may store a variety of types of data for use by controller 220. These types of data may be stored for entire fleets of vehicles as desired. In this embodiment, memory 230 stores vehicle tracking data 232, such as a history of GPS coordinates, or route information (e.g., a route previously taken or yet to be taken). Vehicle tracking data 232 may further include Estimated Time of Arrival (ETA) information (e.g., a specific time of arrival) or an amount of time left before arrival is expected for various vehicles in the fleet that are performing medical transport. Vehicle tracking data 232 may be updated in real-time by crew device in each vehicle in order to provide continuous updates of position and/or status. For example, vehicle tracking data 232 may be updated at each stage of medical transport, such as when vehicle 170 is assigned/dispatched to provide medical transport, when vehicle 170 is en route to the pick up location, when vehicle 170 is at the pick up location, when vehicle 170 is transporting the patient, when vehicle 170 has arrived at the drop off location, and when vehicle 170 is available/clear to provide medical transport for other patients. In further embodiments, crew device 160 provides treatment information to fleet control server 110, indicating medical procedures performed on the patient during medical transport. This treatment information may be transmitted by reporting server 150 to an interested party via mobile device 120, or to provider network 180. As used herein, an interested party may be the patient, or may be an entity distinct from the patient, such as a witness or bystander, a family member, a third-party dispatcher, etc. In some embodiments, a provider of medical services may qualify as an interested party. Crew device 160 may even report a status of the patient in real time to fleet control server 110, and reporting server 150 may transmit the status of the patient to the interested party via mobile device 120, or to provider network 180.

Memory 230 further stores request tracking data 234. Request tracking data 234 may indicate which vehicles have been assigned to perform medical transport based on different requests, the nature of each request, whether a request has been completed (e.g., by the delivery of a patient to a drop off location indicated in the request), a time at which each stage of medical transport was completed for the request, whether a request is presently queued and awaiting assignment of a vehicle, etc.

Still further, memory 230 includes crew data 236. Crew data 236 indicates the efficacy of the crew of the vehicles in the fleet. For example crew data 236 may include survey results for each crew member within a vehicle, such as whether a given crew member is consistently late or on time, whether individual crew members interact positively with each other or staff at medical facilities, which medical qualifications and/or certifications each crew member has, etc.

FIG. 2 further illustrates display 240, such as a monitor or screen operated by controller 220 in order to present aggregated data to a user. For example, display 240 may provide a live map of fleet vehicles that facilitates fleet management and monitoring operations. In further embodiments, controller 220 may provide information for presentation via a display at mobile device 120, CAD system 130, and/or a computer at provider network 180.

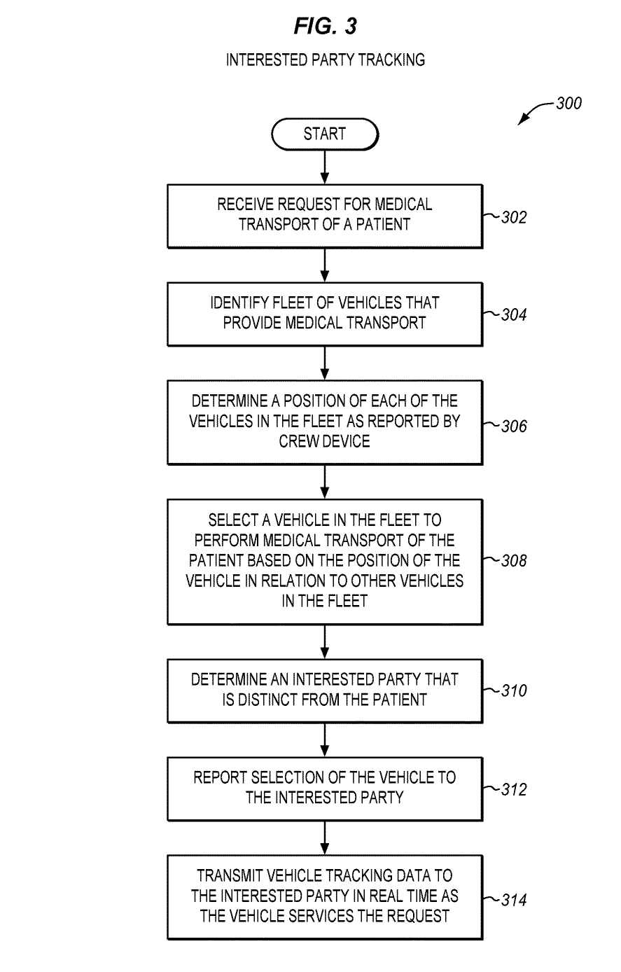

Illustrative details of the operation of medical transport environment 100 will be discussed with regard to FIG. 3. Specifically, FIG. 3 illustrates how interested parties may be kept informed of the progress of a vehicle currently performing medical transport for a patient. Assume, for this embodiment, that a user of mobile device 120 has witnessed a car crash and has determined that a passenger involved in the car crash is in need of medical care. The user of the mobile device 120 therefore determines that there is a need for emergency medical transport of the passenger to a hospital.

FIG. 3 is a flowchart illustrating a method 300 for providing medical transport information to an interested party in an exemplary embodiment. The steps of method 300 are described with reference to medical transport environment 100 of FIG. 1, but those skilled in the art will appreciate that method 300 may be performed in other systems as desired. The steps of the flowcharts described herein are not all inclusive and may include other steps not shown. The steps described herein may also be performed in an alternative order.

The user of mobile device 120 makes a call to an emergency telephone number (e.g., 9-1-1). The call is serviced by dispatch UE 132 at a Public-Safety Answering Point (PSAP), and the user indicates during the call that the passenger may be a patient in need of medical transport. This information is entered into CAD system 130, which generates a request for medical transport of the patient. In some embodiments, the request includes a specific pick up location (e.g., intersection, address, venue, etc.), a specific drop off location, a provider of medical services at the drop off location, and other information describing the status of the patient and/or circumstances relating to the need for medical transport of the patient. The request may also include information describing an age, name, sex, condition, or diagnosis of the patient. The request may even indicate whether it is an explicit request for immediate medical transport, is a request for medical response, or is a request for a scheduled instance of medical transport that takes place at a specific time (or range of times) in the future. The request is transmitted to fleet control server 110 via data network 140.

In step 302, fleet control server 110 receives the request for medical transport of the patient. Fleet control server 110 analyzes the request to identify the pick up location of the patient. Fleet control server 110 further identifies a fleet of vehicles that provide medical transport in step 304. The fleet includes vehicles which are managed by fleet control server 110 during day-to-day operations. For example, the fleet of vehicles may comprise all vehicles that belong to the same company and are presently crewed and ready for medical transport. Fleet control server 110 may detect the number and nature of vehicles within the fleet based on input provided by crew devices within each vehicle. Each crew device may for example report whether its corresponding vehicle is operational and ready for dispatch, may indicate types of care available at its vehicle, may indicate a make and model of its vehicle, may indicate crew qualifications/certifications, and/or a unique identifier of its vehicle. A crew device may also indicate whether its vehicle is already currently engaged in performing medical transport. Each crew device further indicates a position of its vehicle. This position may be reported as a Global Positioning System (GPS) coordinate, street address, etc.

In step 306, fleet control server 110 determines a position of each of the vehicles in the fleet, based on input from crew devices within the fleet. With the position of each vehicle known, fleet control server 110 selects vehicle 170 to perform medical transport of the patient in step 308. The selection of vehicle 170 is based on the position of vehicle 170 with respect to other vehicles. For example, vehicle 170 may be selected because it is the closest vehicle in straight line distance to the pick up location, is expected based on its location to have the shortest time to arrival at the pick up location, or has the shortest route to the pick up location. In some embodiments, fleet control server 110 performs initial filtering steps to reduce the processing burden involved in selecting vehicle 170. For example, fleet control server 110 may subdivide vehicles into predefined geographic areas (e.g., cities, neighborhoods, etc.), and may limit the initial selection process to vehicles which are in the same predefined geographic area as the pick up location.

In further embodiments, fleet control server 110 filters out vehicles which do not provide a required type of care (e.g., BLS, ALS, etc.). For example, fleet control server 110 may identify one or more types of care to be provided during transport of the patient. This information may be provided explicitly in the request, or may be determined dynamically by fleet control server 110 based on symptoms listed in the request. In such an embodiment, fleet control server 110 filters out vehicles which do not have the desired combination of types of care. Fleet control server 110 thus prevents selection of vehicles that are incapable of providing the desired types of care.

With vehicle 170 selected for medical transport, fleet control server 110 determines an interested party that is distinct from the patient in step 310. In this instance, the interested party is the person who called the emergency phone number. However, in further embodiments the interested party may be a family member, a predefined emergency contact for the patient, a provider that will be receiving the patient for treatment, etc. This information may be explicitly listed in the request, or may be determined by fleet control server 110 based on medical records of the patient that are accessible to CAD system 130, provider network 180, and/or fleet control server 110.

With the interested party identified, fleet control server 110 proceeds to compile information that will inform the interested party of the progress of vehicle 170. To this end, fleet control server 110 reports selection of vehicle 170 to the interested party in step 312. This may comprise providing data via an Application Programming Interface (API) to an “app” loaded at mobile device 120, providing a Short Messaging System (SMS) message to mobile device 120, or providing an email or call to mobile device 120 in order to indicate that a vehicle has been dispatched to provide medical transport for the patient.

This information may also indicate medical conditions of the patient indicated in the medical records of the patient, such as whether the patient is currently sick (e.g., has diabetes) or is undergoing treatment. Such information may help the interested party to aid the patient prior to the arrival of vehicle 170. Depending on whether a waiver has been signed by the patient, provisioning of sensitive medical data about the patient may be prohibited by fleet control server 110 in order to comply with the requirements of the Health Insurance Portability and Accountability Act (HIPAA).

Fleet control server 110 additionally tracks the location of vehicle 170 in real time based on input from crew device 160. Fleet control server 110 may further update an ETA or Time to Arrival (TTA) of vehicle 170 based on this information. In one embodiment, fleet control server 110 generates a map illustrating a position of vehicle 170 with respect to the interested party, the patient, the pick up location, and/or the drop off location. Reporting server 150 further transmits vehicle tracking data (e.g., GPS coordinates, speed, direction, etc.) to mobile device 120 in real time as vehicle 170 services the request (step 314). In this manner, the interested party may view the progress of vehicle 170 in real time as vehicle 170 moves towards the pick up location. As used herein, “real time” may refer to actual real time (e.g., updates that are up-to-date within a second) or near-time (e.g., updates that are up to five to ten seconds apart).

In one embodiment, fleet control server 110 coordinates with a third party mapping server (such as a server for Google Maps, Bing Maps, etc.), to provide GPS coordinates over time to the mapping server. The mapping server may in turn generate a link to a map illustrating progress of vehicle 170 over time. Reporting server 150 may provide this link to mobile device 120 as a form of vehicle tracking data.

After a period of time, vehicle 170 arrives at the pick up location, and an updated status of vehicle 170 is determined by fleet control server 110. For example, a crew member at vehicle 170 may indicate a change in status via a touch screen at crew device 160, which triggers transmission of an update from crew device 160 to fleet control server 110. A change in status and/or location of vehicle 170 may alternatively be reported to fleet control server 110 via CAD system 130. Changes in vehicle status may occur when the patient is picked up, transported, taken to the drop off location, and/or dropped off. In some embodiments, fleet control server 110 infers an updated status for vehicle 170 based on a proximity of vehicle 170 to the pick up or drop off location. For example, fleet control server 110 may automatically update the status of vehicle 170 to “pick up” or “drop off” when vehicle 170 is within one hundred yards of the pick up location or drop off location. This updated information may be provided to the interested party (via mobile device 120), to provider network 180, and/or to the dispatcher (via CAD system 130) as desired. After drop off has been completed, crew device 160 may update its status to indicate that the request has been fully serviced.

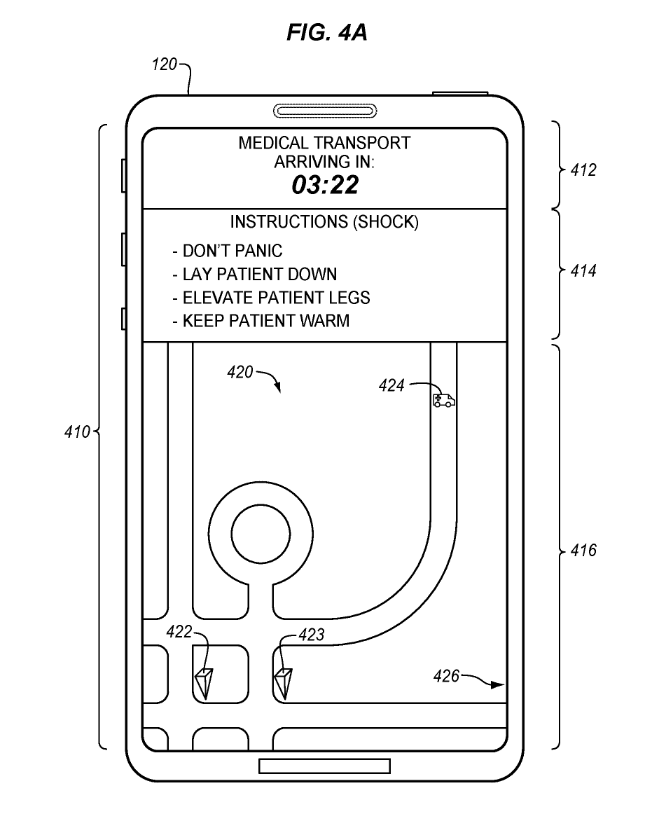

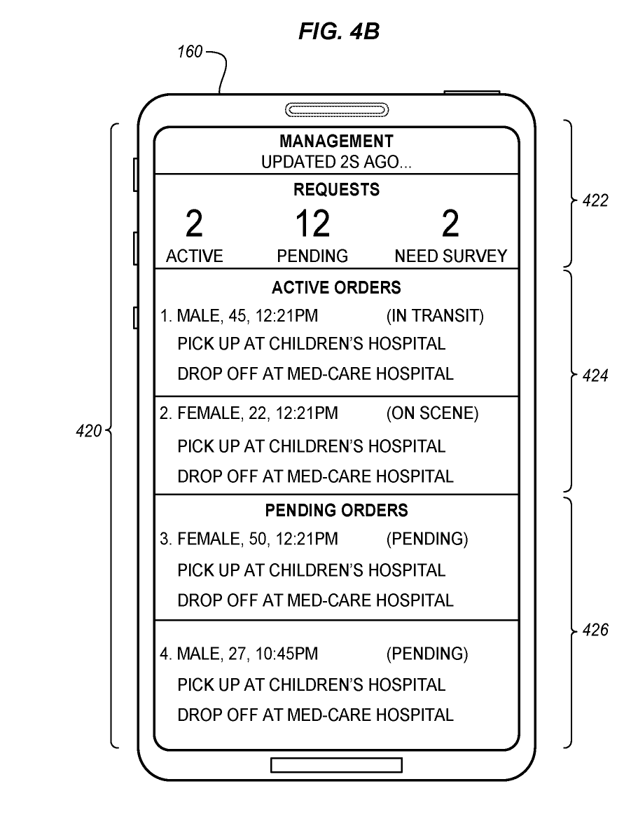

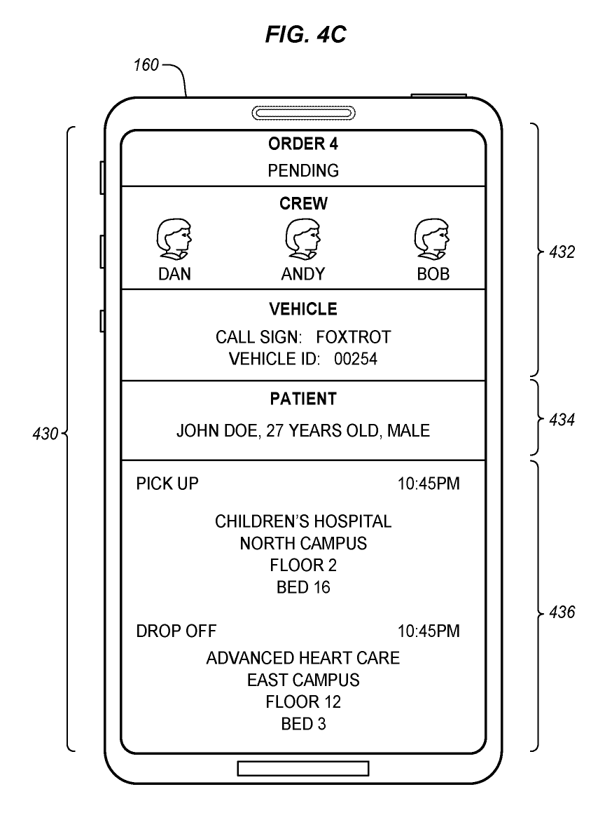

FIGS. 4A-4C are diagrams illustrating information presented at various devices in exemplary embodiments. For example, FIG. 4A is a diagram illustrating medical transport information presented on the display of mobile device 120 of an interested party in an exemplary embodiment. As shown in FIG. 4A, display 410 is divided into several different areas. Area 412 presents an arrival time estimate in the form of a countdown timer. Area 414 presents bystander instructions to the interested party for treating a medical condition of the patient (e.g., shock). The bystander instructions may allow for the interested party to initiate first aid or other treatment of the patient, increasing the chances of successful recovery by the patient. In further embodiments, the instructions provide general guidelines for the interested party that are applicable regardless of the medical condition of the patient. An example of such an instruction would be “stay out of the street,” or “do not leave the scene.”

Area 416 presents map 420. Map 420 includes an icon 424 representing vehicle 170, which is en route to the pickup location 422 (e.g., a location of the patient). Location 423 of the interested party (e.g., as reported by mobile device 120) is also provided on map 420. In this embodiment, map 420 further includes indicator 426, which may point to an off-map location (e.g., a drop off location corresponding with a provider that will treat the patient, the location of a back up vehicle, etc.).

FIG. 4B is a diagram illustrating medical transport information presented on a display of crew device 160 of vehicle 170 in an exemplary embodiment. This information may be presented in response to a crew member at vehicle 170 wishing to browse incoming requests for medical transport. As shown in FIG. 4B, an overview portion 422 of screen 420 describes a number of active requests currently being serviced by one or more vehicles, a number of pending requests yet to be serviced, and a number of requests that have been serviced and are awaiting feedback. Meanwhile, portion 424 of screen 420 illustrates active orders being handled by one or more vehicles, and portion 426 illustrates pending order for which no vehicle has yet been assigned.

FIG. 4C is a diagram illustrating further medical transport information presented on a display of crew device 160 of vehicle 170 in an exemplary embodiment. This information may be presented, for example, in response to a user of crew device 160 tapping on the bottom request of FIG. 4B. As shown in FIG. 4C, screen 430 includes overview information in section 432. This information identifies a status of the request (“pending”), the crew assigned to service the request, and the vehicle assigned to provide medical transport for the request. Meanwhile, portion 434 provides patient information (which may include current symptoms of the patient). Portion 436 provides pick up and drop off information, including expected times of arrival at specific locations, and the exact nature of the pick up and drop off locations. Further discussion of the specific communications involved in coordinating medical transport is provided below.

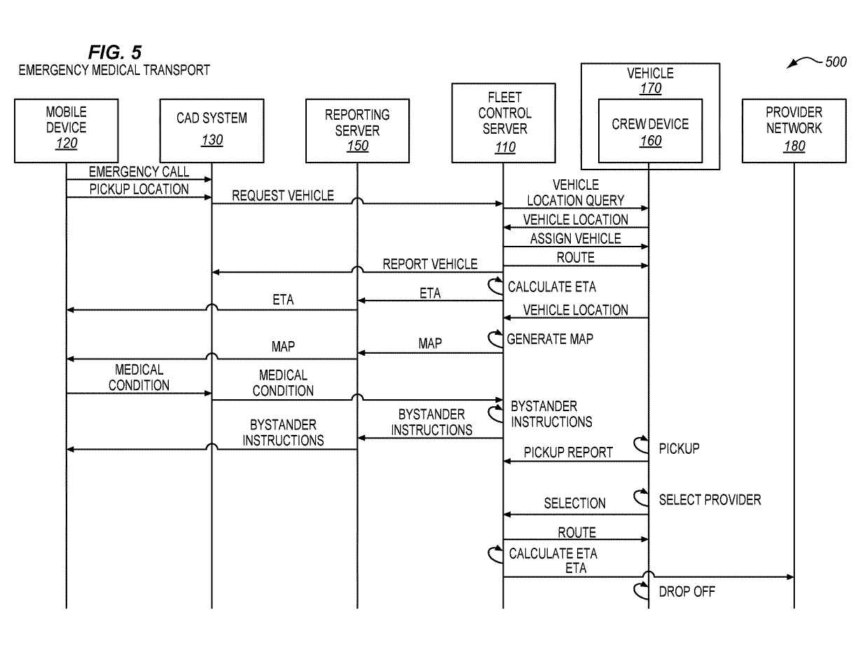

FIG. 5 is a message diagram 500 illustrating communications for emergency medical transport in an exemplary embodiment. Specifically, FIG. 5 illustrates interactions between the various components and devices of FIG. 1 during an instance of emergency medical transport. According to FIG. 5, communications may initiate when mobile device 120 initiates an emergency call to a PSAP via cellular network 142, which is recorded at CAD system 130. The emergency call may be accompanied by information describing a pick up location (e.g., a GPS coordinate of mobile device 120 when the call is initiated, an address indicated by the caller, etc.). CAD system 130 submits an electronic request for medical transport to fleet control server 110. For example, CAD system 130 may submit the request via data network 140 using an API supported by fleet control server 110. In further embodiments, electronic requests and/or emergency calls relating to medical transport may be generated by an application at mobile device 120 in communication with fleet control server 110, by provider 180, or by any suitable electronic component (e.g., including components utilizing landlines).

Fleet control server 110 receives the request, identifies the pick up location, and queries fleet vehicles (including vehicle 170) for their locations in order to determine the best available vehicle for servicing the request. In further embodiments, fleet control server 110 may receive calls directly from mobile device 120 and internally generate its own requests.

In this embodiment, fleet control server 110 selects the closest fleet vehicle which is in service, not presently performing medical transport, and closest to the pickup location. The vehicle meeting these criteria is vehicle 170. Upon selecting vehicle 170, fleet control server 110 transmits an assignment message to vehicle 170 indicating the details of medical transport to provide. In this embodiment, fleet control server 110 also calculates and transmits route information indicating a path for vehicle 170 to follow in order to reach the pick up location. The assignment message may also include details regarding the medical condition of the patient, types of care to be provided to the patient during medical transport, and other relevant information reported by CAD system 130.

Fleet control server 110 reports the assignment of vehicle 170 to CAD system 130, calculates an arrival time estimate (in this case, ETA) for vehicle 170, and transmits the arrival time estimate to reporting server 150 via data network 140. Reporting server 150 transmits the arrival time estimate to mobile device 120, for example via an API for an application at mobile device 120, via SMS message, or via telephone call. This allows the interested party to be informed of the expected pick up time.

In this embodiment, fleet control server 110 generates additional information which may be of use to the interested party, and provides that information to mobile device 120 via reporting server 150. Specifically, fleet control server 110 coordinates generation of a map (e.g., including the location of vehicle 170, the interested party, a pick up location, and/or a drop off location), and provides the map to mobile device 120 via reporting server 150 (e.g., as a Uniform Resource Locator (URL)). Fleet control server 110 further determines a medical condition of the patient (e.g., shock, concussion, etc.) based on input from CAD system 130. The medical condition may be determined, for example, based on a keyword provided in the message from CAD system 130. Fleet control server 110 proceeds to acquire bystander instructions (e.g., as stored in memory) that are associated with the medical condition, and provides the bystander instructions to the interested party via reporting server 150 and mobile device 120 in order to facilitate treatment. In further embodiments, bystander instructions may be provided based on the type of incident that caused a need for medical transport, such as based on whether the interested party is close to a car crash, downed power line, fire, etc.

After some period of time, vehicle 170 arrives at the pick up location to pick up the patient. A crew member of vehicle 170 generates a pick up report via crew device 160, and crew device 160 transmits the pick up report to fleet control server 110 for analysis. The pick up report may include a current status of the patient and/or vehicle 170, any changes of symptoms, a time and/or position at which pick up was performed, etc.

Crew at vehicle 170 may further select a provider to perform medical services to provide treatment of the patient, and reports this selection to fleet management server 110. Based on the provider selected, fleet management server 110 determines a drop off location for the patient. For example, the drop off location may be an entrance to a medical facility of the provider. In further embodiments, a provider may be automatically selected by fleet control server 110 based on received information describing types of care needed by the patient. For example, a patient suffering from burns may be directed to a burn unit of a provider.

Based upon the selected provider, fleet control server 110 may create a route provided for traveling from the pick up location (or current location of vehicle 170) to the drop off location, may calculate an arrival time estimate for arriving at the current provider, and/or may even suggest selection of another provider (e.g., a provider that is closer). Fleet control server 110 further transmits the arrival time estimate to provider network 180 (or other additional interested party). Fleet control server may additionally generate and transmit a map to provider network 180 if desired. Upon arrival of vehicle 170, fleet control server 110 receives a drop off report from crew device 160 of vehicle 170, which may include similar information to that of the pick up report.

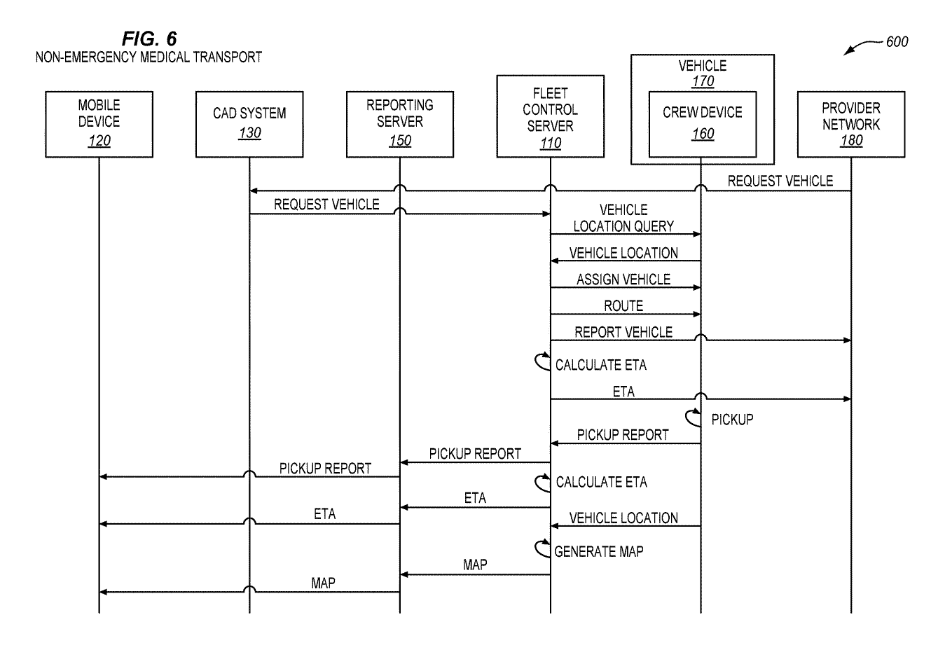

FIG. 6 is a message diagram 600 illustrating communications for non-emergency medical transport in an exemplary embodiment. FIG. 6 includes similar communications to those discussed in FIG. 5. However, in FIG. 6, the request for medical transport is initiated by provider network 180 (e.g., at the pick up location), and the interested party receives an ETA for drop off (e.g., because the interested party is at a drop off location). The drop off location may be determined beforehand, for example by provider network 180. In further embodiments, requests for medical transport may be initiated by the interested party via mobile device 120, via a web application, or any other suitable component (e.g., via direct communication with fleet control server 110, or based on communications with CAD system 130 via a call to a dispatcher).

In further embodiments, requests for medical transport may be initiated by mobile device 120 or a computer at provider network 180 interacting with an application that provides for direct interaction with fleet control server 110.

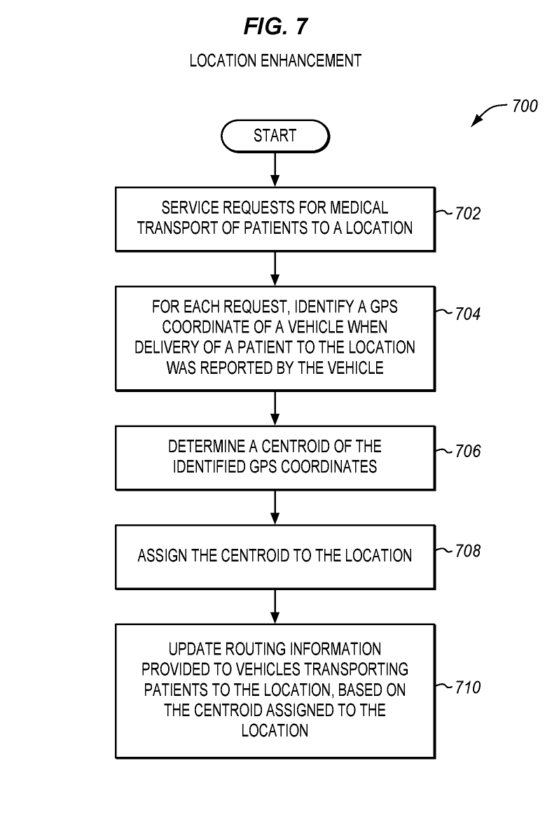

The techniques discussed with regard to FIGS. 3-6 keep a variety of parties informed as to the progress of a vehicle providing medical transport. In contrast, FIGS. 7-8 provide techniques that fleet control server 110 may utilize to enhance the precision by which vehicles are routed to common pick up locations and/or drop off locations. Specifically, FIGS. 7-8 illustrate that fleet control server 110 may aggregate information received across numerous pick up reports and/or drop off reports in order to precisely determine the GPS coordinates of various pick up and drop off locations.

Requests for medical transport may use an address to define a pick up location or drop off location. However, a single address may describe the location of a small clinic, or even a large hospital. Hence, if a patient is being dropped off at a surgical center of a large hospital, the intended drop off location may be hundreds of yards distant from a psychiatric ward having the same address at the same hospital. This presents a problem because it results in inefficient “last mile” transport of patients to and from a large medical complex. The lack of precision in location is aggravating because speed is often critical during instances of medical transport.

By enhancing the precision at which common drop off and pick up locations are identified, fleet control server 110 reduces the amount of time spent searching for specific entrances and/or exits to large medical complexes, which in turn means that fleet vehicles waste less time per instance of medical transport. With time being utilized more efficiently, the fleet may transport more patients throughout the day.

FIG. 7 is a flowchart illustrating a method 700 for engaging in location enhancement for medical transport in an exemplary embodiment. According to FIG. 7, fleet control server 110 directs fleet vehicles to service requests for medical transport of patients to a location (e.g., a combination of address and textual identifier) in step 702. As requests are processed over time, some requests will be directed to the same location, such as an address for a hospital, combined with a textual descriptor/identifier for a portion of the hospital. For example, multiple requests may indicate an address of 105 W Cherry St, and be accompanied by the textual descriptor of “Children's Wing” in order to indicate that patient drop off should be performed at the children's wing of the listed hospital.

In step 704, for each request directed to the location, fleet control server 110 identifies a GPS coordinate reported by a vehicle when the vehicle dropped off a patient at the location. For example, fleet control server 110 may identify a GPS coordinate reported by crew device 160 when the drop-off report was completed (or when drop off was otherwise confirmed via crew device 160). In one embodiment, if a fleet vehicle reports drop off while the vehicle is in motion (e.g., moving at several miles per hour or faster), the GPS coordinate may be discarded. This is because the vehicle is still moving, and hence either is still proceeding towards the drop off location, or has already left the drop off location.

After a predefined time period (e.g., a number of days, weeks, months, etc.), or a predefined number of visits (e.g., twenty, one hundred, etc.) to the drop off location by vehicles in the fleet, a substantial number of GPS coordinates are associated with the drop off location. Fleet control server 110 may therefore utilize the aggregated GPS coordinates to precisely determine the “real world” position of the drop off location. To this end, in step 706 fleet control server 110 determines a centroid of the GPS coordinates. The centroid indicates the most likely real-world position of the drop off location, and may be calculated as an average of latitudes, longitudes, and/or elevations of non-outlier GPS coordinates identified in step 704. Fleet control server 110 further assigns the centroid to the drop off location as a GPS coordinate in step 708. Based on the centroid, fleet controls server 110 updates routing information provided to vehicles transporting patients to the location in step 710. For example, an elevation of the centroid may be translated into a floor number of the hospital, and this information may be provided to vehicles transporting patients to the drop off location. The elevation information may be particularly useful to calculate when an MDT of the vehicle exits the vehicle and travels with the crew as the crew move the patient to the appropriate floor within the hospital.

If the centroid is at a different position along a road (e.g., a private road at the hospital) than indicated by the address, or is located at a different road with respect to a GPS coordinate indicated by the address, routing information may be updated to direct incoming vehicles to the appropriate road (or portion thereof). While the techniques of FIG. 7 are described with regard to drop off locations, they may also be performed in order to enhance the precision by which pick up locations are defined (e.g., based on the analysis of GPS coordinates received when pick up reports are generated).

To facilitate the method of FIG. 7, fleet control server 110 may identify “hot spot” locations which are visited at least a threshold number of times over a time period (e.g., ten times a month), and perform enhancement for those locations. In further embodiments, fleet control server 110 may perform separate enhancements for locations that have the same address, but a different textual descriptor. For example, an address of “105 W Cherry St.” for a hospital may be considered a different location when accompanied by the textual descriptor of “psychiatric ward” than when accompanied by the textual descriptor of “Children's Wing.” In still further embodiments, fleet control server 110 engages in location enhancement when vehicles regularly arrive at a GPS coordinate for an address of the drop off location, yet take more than a predefined amount of time (e.g., five minutes, ten minutes) or distance (e.g., one hundred yards) after arriving before they report that drop off has actually been completed.

A visual depiction of the process of FIG. 7 is provided in FIG. 8. FIG. 8 is a diagram illustrating a window 800 utilized for location enhancement in an exemplary embodiment. In FIG. 8, the location being enhanced is the “Children's Wing” drop off location for a hospital, which is indicated via an address (“105 W. Cherry St.) and textual descriptor (“Children's Wing”). In this embodiment, the street address is associated with GPS coordinate 802. However, vehicle crews have complained that GPS coordinate 802 is inaccurate, because it is located several hundred yards from the patient entrance of the children's wing. Based on this information, fleet control server 110 proceeds to engage in location enhancement by reviewing GPS coordinates previously reported by vehicles when those vehicles dropped off patients at the children's wing. These GPS coordinates include GPS coordinates 804 and GPS coordinates 810.

As a preliminary matter, GPS coordinates 804 are located more than a predefined number of standard deviations (e.g., two standard deviations) from other GPS coordinates with respect to latitude and/or longitude. Hence, fleet control server 110 discards GPS coordinates 804 as outliers. Centroid 820 is then calculated based on the remaining GPS coordinates 810, for example as a mean, median, or mode of GPS coordinates 810. In one embodiment, the value of each GPS coordinate 804 is weighted when calculating the centroid, based on an accuracy of that GPS coordinate. Fleet control server 110 assigns centroid 820 to the drop off location for patients at the children's wing. Centroid 820 is then utilized when routing vehicles to the drop off location. As mentioned above, in some embodiments the drop off location is within a building (e.g., on a specific floor of a building). Thus, centroid 820 may also be calculated based on an average of elevation, in addition to being based on an average of latitude and longitude.

In further embodiments, the drop off location for the children's wing may be different from the pick up location for the children's wing, even though both are referred to by a CAD system as “Children's Wing.” Thus, the same “location” may refer to different pick up and drop off coordinates. To address this issue, fleet control server 110 may separately engage in location enhancement for drop off locations and pick up locations, even when incoming requests use the exact same language to refer to such locations. In this example, a patient pick up entrance is indicated by GPS coordinate 806, while a visitor entrance is indicated by GPS coordinate 830.

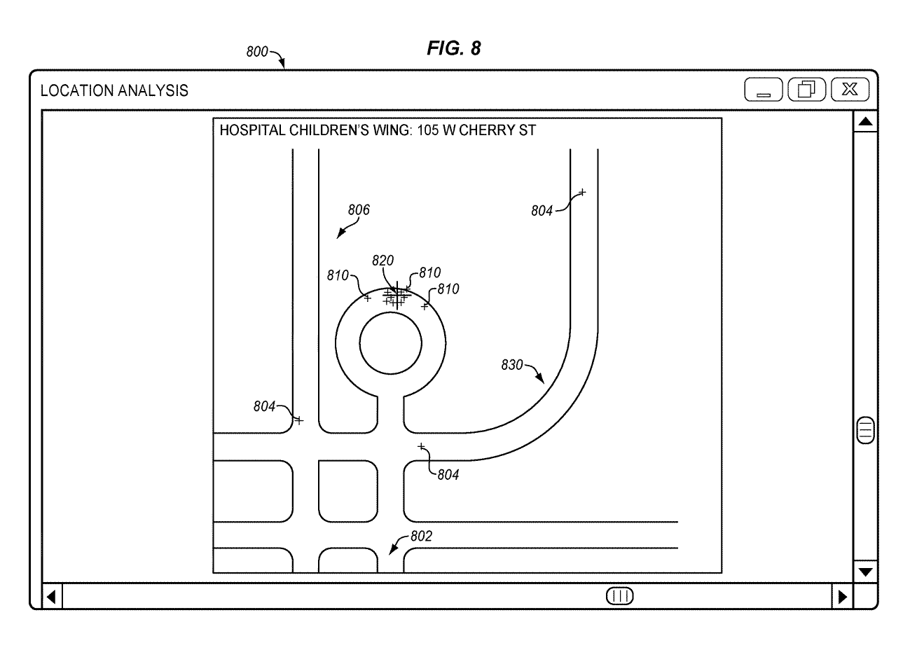

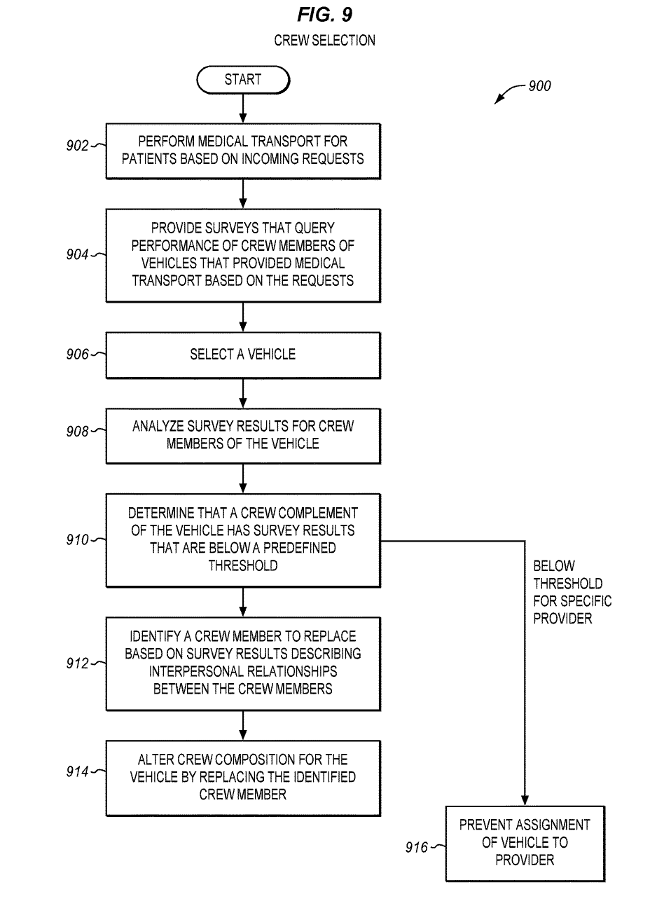

FIGS. 9-11 illustrate techniques by which fleet control server 110 may be utilized in order to revise the crew of a fleet vehicle, based on interactions of the crew members with each other and/or personnel at a provider of medical services. For example, FIG. 9 is a flowchart illustrating a method for revising the crew of a medical transport vehicle in an exemplary embodiment. According to FIG. 9, fleet control server 110 directs fleet vehicles to perform medical transport based on incoming requests in step 902. In step 904, at the completion of each pick up and/or drop off, fleet control server 110 provides a survey that queries the performance of crew members. That is, each time a vehicle picks up or drops off a patient, fleet control server 110 may provide surveys for rating the crew members of that vehicle. These surveys may be provided to an interested party via mobile device 120, to one or more staff members at a provider of medical services, and/or to each crew member in the vehicle via crew device 160. The survey results may then be transmitted to fleet control server 110 for analysis. In one embodiment, a separate pick up survey, drop off survey, and/or crew survey is provided for each instance of medical transport. Each survey may be associated with a different crew, or even a different individual crew member. For example, a first combination of crew members for a vehicle may be associated with a first crew ID, while a second combination of crew members may be associated with a second crew ID. Individual surveys may then each be associated with the ID of the crew that performed the related instance of medical transport. Surveys may request any suitable combination of qualitative and quantitative information regarding the instance of medical transport.

After a predefined number of surveys have been collected for a vehicle, or after the vehicle has operated for a predefined amount of time (e.g., a month), fleet control server 110 selects the vehicle for analysis in step 906. Fleet control server 110 further analyzes survey results for crew members of the vehicle in step 908. For example, fleet control server 110 may analyze survey results for each crew member, based on responses from other crew members and/or staff at submitting survey responses via provider network 180. Based on the survey results, in step 910 fleet control server 110 determines whether the crew of the vehicle have survey results that are below a predefined threshold (e.g., three of five stars). If the crew is below the threshold, then fleet control server 110 proceeds to identify a crew member to replace based on survey results describing interpersonal relationships between the crew members in step 912. For example, if a crew member has negative relations with other crew members, this may be inferred to be the source of the negative survey results. Hence, fleet control server 110 proceeds to alter crew composition for the vehicle by replacing the identified crew member in step 914. In this manner, if a crew is experiencing quality issues, re-assignment of the crew member to a new vehicle may ensure that standards of quality are met. In a further embodiment, survey information may be utilized by fleet control server 110 to ensure that the vehicle does not service requests for medical transport from providers that rate crew members of the vehicle below a certain threshold. Thus, in circumstances where relations with a specific provider are consistently low, after step 910 fleet control server 110 prevents the vehicle from being assigned to requests for medical transport relating to that provider (step 916).

In further embodiments, fleet control server 110 may additionally and/or alternatively identify conflicts between crew members and medical staff at one or more provider. For example, if a crew has consistently positive survey results with most providers, but has negative reviews from one provider, the vehicle for that crew may be flagged as “unavailable” when selecting vehicles from the fleet to provide medical transport to the provider. This flag may be set so that it only applies to non-emergency transport, if desired.

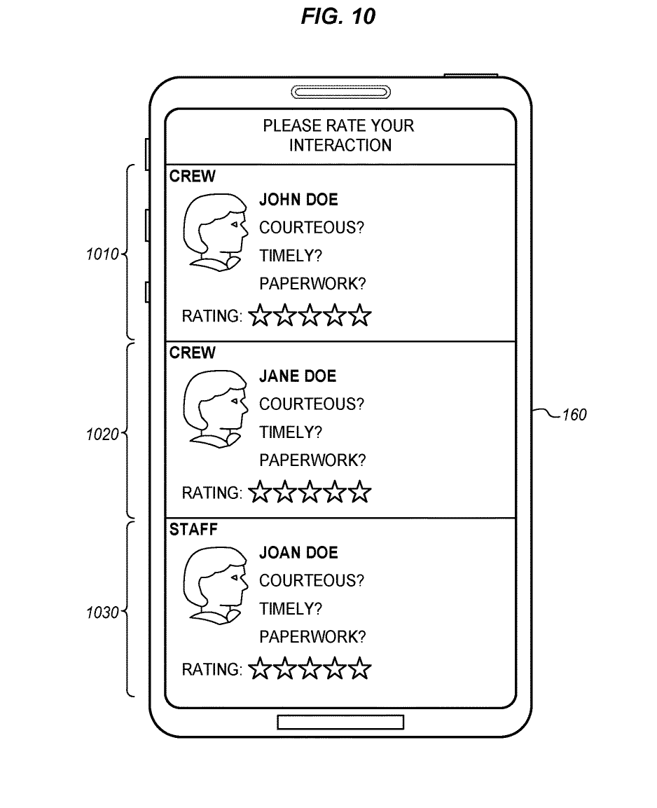

FIG. 10 is a diagram illustrating a display utilized for reporting satisfaction with crew for a medical transport vehicle in an exemplary embodiment. As shown in FIG. 10, a survey presented at crew device 160 includes questions pertaining to each crew member in sections 1010, 1020, and 1030, respectively. These questions may vary based on the training and/or role of each crew member. For example, questions for a paramedic may relate to quality of treatment during medical transport, while questions for an Emergency Medical Technician (EMT) may be more general in nature. In addition to one or more specific questions, a star rating is provided for rating that crew member. This survey may be provided to each crew member, to medical staff via provider network 180, or to one or more interested parties. These survey results may be utilized as input for method 900 of FIG. 9, or may be used by fleet control server 110 to report issues with medical staff to provider network 180. For example, if a member of medical staff is consistently late with discharge paperwork, resulting in a delay in patient pick up, fleet control server 110 may identify this problem and provide a recommendation for resolving the issue to provider network 180 via data network 140.

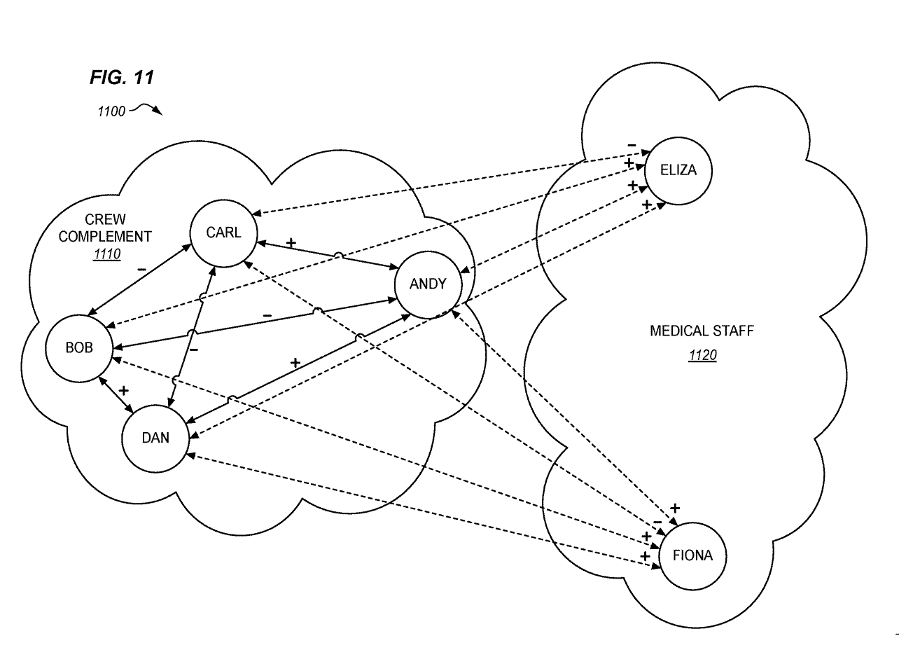

FIG. 11 is a social graph illustrating relationships between crew members of a medical transport vehicle and medical staff in an exemplary embodiment. In this embodiment, a vehicle has a crew that includes Andy, Bob, Carl, and Dan. These crew members interact with medical staff 1120, in this case Eliza and Fiona. Fleet control server 110 analyzes survey results between the various personnel involved in medical transport at the vehicle, and determines that both Bob and Carl each have two negative relationships with other crew members. Fleet control server 110 further determines that Carl also has negative relationships with medical staff, while Bob has positive relationships with the medical staff. Thus, fleet control server 110 determines that Carl should be re-assigned in order to enhance relationships between crew members, as well as to enhance interactions with medical staff 1120. The re-assignment of crew may therefore be based not just on interactions between crew members, but also based on interactions between crew members and personnel at a provider of medical services. In a further embodiment, instead of re-assigning Carl, fleet control server 110 prevents Carl's vehicle from be assigned to service requests from the provider that has negative interactions with Carl.

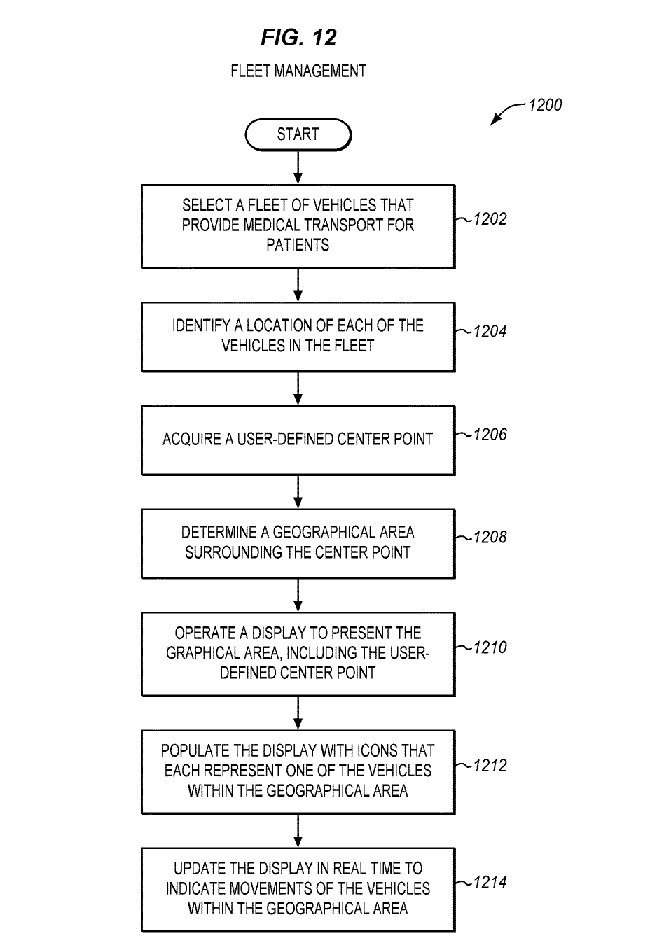

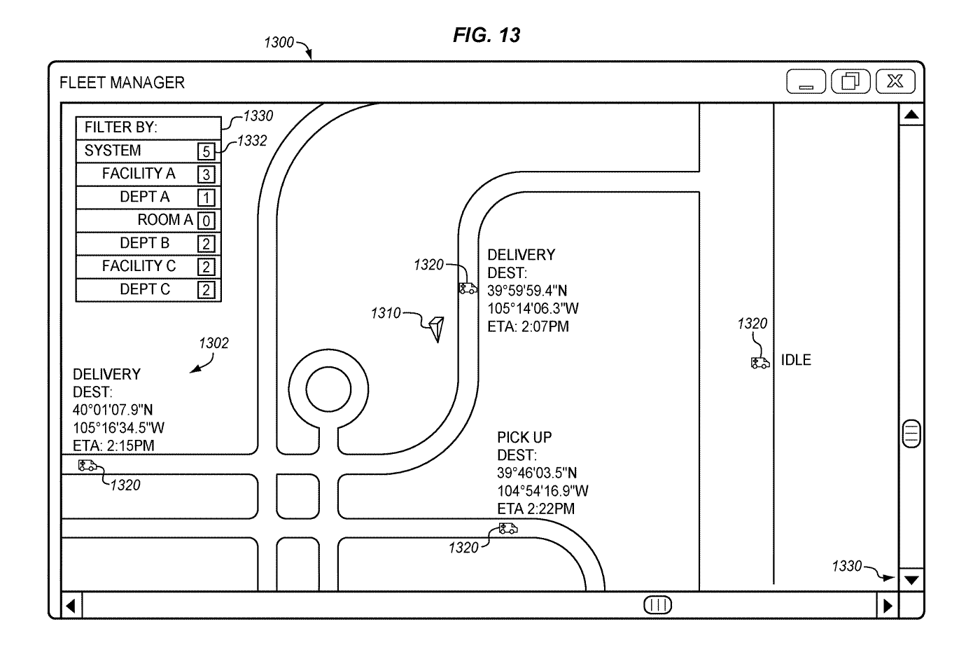

FIGS. 12-13 illustrate a further embodiment where a user of fleet control server 110 monitors the movement of a fleet of vehicles. Specifically, FIG. 12 is a flowchart illustrating a method for selecting performing fleet management for medical transport services in an exemplary embodiment. Assume, for this embodiment, that fleet control server 110 manages multiple fleets of vehicles, and that a user has logged into fleet control server 110 in order to manage one of those fleets. According to FIG. 12, in step 1202 fleet control server 110 selects a fleet of vehicles that provide medical transport for patients. The fleet may be selected based on the credentials of the user. For example, in embodiments wherein fleet control server 110 manages multiple fleets of vehicles, each fleet corresponding with a different company, fleet control server 110 may determine the company which the user is associated with, and display only the fleet of vehicles used by that company.

In step 1204, fleet control server 110 identifies a location of each of the vehicles in the fleet. This may be provided, for example, as a periodic update from MDTs at the vehicles. Fleet control server 110 further proceeds to acquire a user-defined center point in step 1206. This may be included, for example, in a profile for the user stored in memory. The center point may be defined as an address, GPS coordinate, etc. An example of a center point may be a headquarters or dispatch center address of the company. Based on the user-defined center point, fleet control server 110 determines a geographic area surrounding the center point in step 1208. For example, the geographic area may be a radius around the center point, or may even be the boundaries of a medical network, campus, facility, department, floor, etc. The geographic area may be centered on the center point, or may be offset while still including the center point. For example, for a coastal center point, the geographic area surrounding the center point may be offset by a predefined amount (e.g., twenty miles landward) in order to increase the amount of land mass shown. Fleet control server 110 further proceeds to operate a display (e.g., display 240, a display at mobile device 120, etc.) to present the graphical area, including the user-defined center point, in step 1210.

In step 1212 fleet control server 110 populates the display with icons that each represent one of the fleet vehicles within the geographical area. Fleet control server 110 additionally updates the display in real time to indicate movements of the vehicles within the geographical area in step 1214.

User preferences may also indicate what type of information to display on the map (e.g., patient information, vehicle speed and orientation, information specific to the request, etc.), whether the map is dynamically zoomed in and out based on the movements of one or more fleet vehicles, and a variety of actions to take after completion of an instance of medical transport. These actions may indicate how long to continue presenting a survey to involved parties after the patient has been transported, whether to return the map to a “default” zoom level and location after the patient has been transported, etc.

FIG. 13 is a diagram illustrating a window 1300 utilized for fleet management of medical transport services in an exemplary embodiment. For example, window 1300 may be part of an administrative interface that enables a user to configure a customized, zoomable view of the geographical area, a history/log of medical transport for a fleet of vehicles, historical survey results for a crew member or entire crew, etc. Such views may be useful for users who manage a fleet of vehicles, providers of medical services that wish to track incoming and/or outgoing instances of medical transport to their facilities, and others.

In this embodiment, window 1300 includes map 1302, user-defined center point 1310, and multiple icons 1320 (one for each vehicle). Icons 1320 may vary in size, shape, or color depending on the type(s) of care available at each vehicle. Furthermore customizable contextual data is presented with each icon 1320, in order to indicate a location, ETA, speed, etc. of each of the vehicles. Vehicles that are beyond the geographical area may be indicated via indicators 1330 located at the edge of window 1300.

A user of window 1300 may further view, add, edit, re-assign or delete requests for medical transport. For example by clicking on a vehicle, the user may view the medical transport being provided by that vehicle. The user may then revise the request based on input from CAD system 130 or an interested party, or may even assign the request to a different vehicle. In this embodiment, a user may center their view in real time to follow a vehicle by clicking on an icon 1320 representing that vehicle.

FIG. 13 further includes a user interface 1330, which enables selection control, and/or filtering of vehicles displayed at window 1300. For example, a provider of medical services may utilize user interface 1330 to filter out vehicles that are picking up (or dropping off) patients at a specific medical system, facility, campus, building, department, floor, or even room. When utilized by a dispatcher, window 1300 may include multiple facilities. In contrast, users at a facility may desire to view a more limited area. In this embodiment, user interface 1330 further includes clickable elements 1332, which each report a number of requests pending for a system, facility, department, or room. By clicking on an element 1332, pending requests may be selected or de-selected for display at window 1300.

In further embodiments, permission-based access may be used to enable medical staff at a provider of medical services (or interested parties) to view vehicles from the fleet which have been scheduled to engage in medical transport to or from a corresponding medical facility. This information may include request information, a status of the vehicle, a location of the vehicle on an interactive map, as well as arrival and departure time estimates. The number of vehicles shown may be filtered based on, for example, the hospital network that the medical transport relates to, or may be defined on a more granular level such as campus, facility, department, floor or even a single patient.

### EXAMPLES

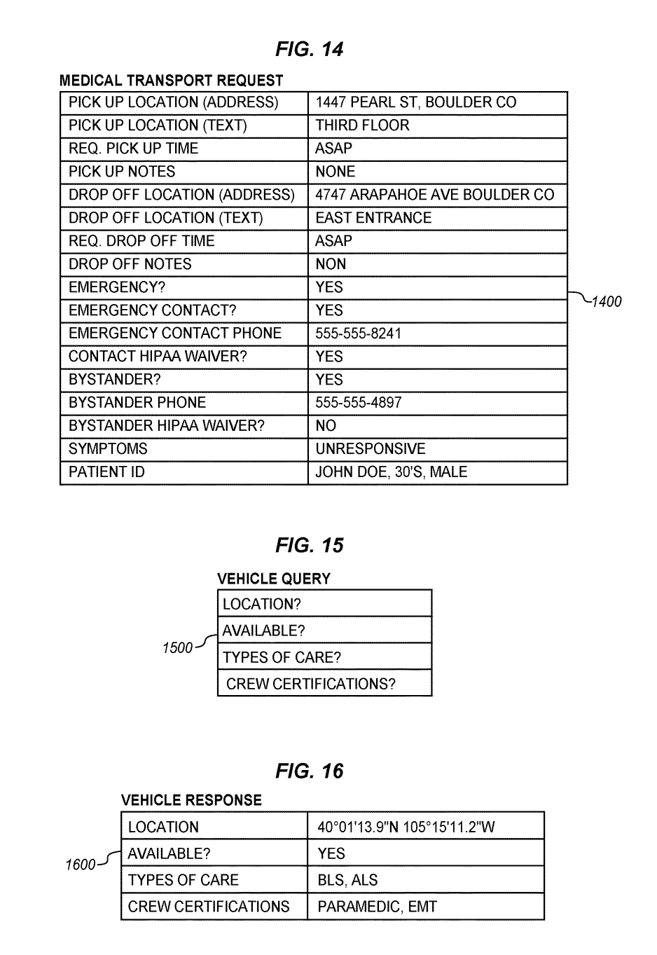

In the following examples, additional processes, systems, and methods are described in the context of messaging sent to or from fleet control server 110. Specifically, FIGS. 14-18 illustrate various messages exchanged within a medical transport environment in an exemplary embodiment. These messages may be exchanged via any suitable messaging format, such as via JavaScript Object Notation (JSON). While the various fields described herein are illustrated as being populated with specific values, in further embodiments a field may be populated with a pointer to an entry in a database stored on fleet control server 110.

FIG. 14 illustrates an exemplary request 1400 for medical transport provided to fleet control server 110. In this example, request 1400 is provided via packetized communications, and comprises a series of named fields, each named field being paired with a value. Specifically, this example utilizes named Extensible Markup Language (XML) fields to delineate different pieces of information. Request 1400 includes an address for a pickup location, a textual description providing additional details regarding the pick up location, listed pick up notes, a requested pick up time, an address and textual description for a drop off location, drop off notes and a requested drop off time. Request 1400 further includes an “emergency” field, which is a binary flag that indicates whether the requested instance of medical transport relates to an emergency or not. Request 1400 also indicates whether the patient has a known emergency contact, a phone number of the emergency contact, and a field indicating whether the patient has a HIPAA waiver on record allowing the transmission of personal information to the emergency contact. Fields are also provided indicating whether a bystander is present, contact information for the bystander, and whether the patient has a HIPAA waiver on record allowing the transmission of personal information to bystanders. A list of symptoms is also provided, as well as identifying information describing the patient, such as by name, age, sex, etc.

Based on request 1400, fleet control server 110 provides a query 1500 to each vehicle in the fleet (as shown in FIG. 15). Query 1500 requests a precise vehicle location, an indication of whether the vehicle is presently available, a list of types of care provided by the vehicle, and a list of crew qualifications for the vehicle. In response to query 1500, each vehicle may provide a response, such as response 1600 of FIG. 16. In this example, response 1600 provides a coordinate describing a location of the vehicle, a binary flag indicating whether the vehicle is available, a list of types of care provided by the vehicle, and a list of crew certifications for a crew of the vehicle.

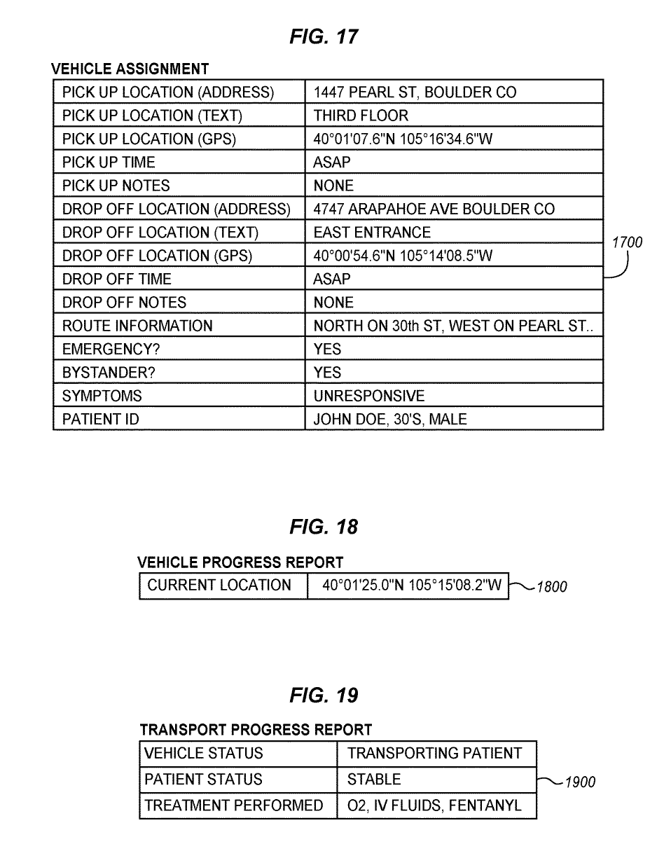

Based on this information, fleet control server 110 selects vehicle 170 to provide the medical transport. Fleet control server 110 therefore provides a vehicle assignment message 1700 (FIG. 17) to vehicle 170 via crew device 160. In this example, vehicle assignment message 1700 includes a street address, textual description, GPS coordinate, pick up notes, and pick up time for pick up location, and includes similar information for drop off location. The GPS coordinates are location enhanced GPS coordinates as described above, instead of GPS coordinates for the listed address. This helps to ensure that vehicle 170 will arrive at a desired pick up or drop off location, instead of just a street address nearby the desired pick up or drop off location. Vehicle assignment message 1700 further includes routing information provided by fleet control server 110, an indicator as to whether the medical transport relates to an emergency, whether a bystander is presently at the scene, a list of symptoms, and identifying information about the patient, such as name, sex, age, race, etc.

As vehicle 170 proceeds towards the pick up location, or as vehicle 170 proceeds towards the drop off location, crew device 160 may provide vehicle progress reports in real-time to fleet control server 110 as shown in FIG. 18. For example, vehicle progress report 1800 may be provided while the patient is being transported. In this example, vehicle progress report 1800 includes a coordinate for the location of vehicle 170. In addition to vehicle progress reports, crew device 160 may provide user-generated (or automatically generated) transport progress reports 1900 (as shown in FIG. 19), which indicate a status of the vehicle during transport, a status of the patient, and/or a list of medical treatments provided to the patient during transport.

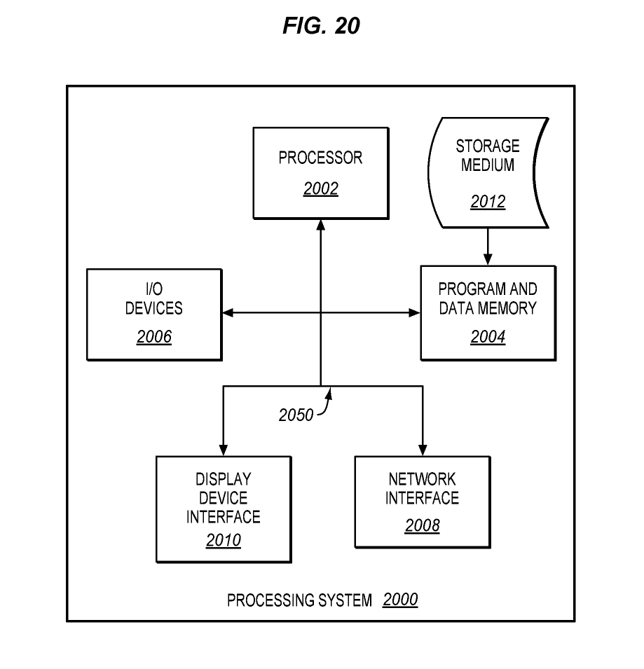

Embodiments disclosed herein can take the form of software, hardware, firmware, or various combinations thereof. In one particular embodiment, software is used to direct a processing system of fleet control server 110 to perform the various operations disclosed herein. FIG. 20 illustrates a processing system 2000 operable to execute a computer readable medium embodying programmed instructions to perform desired functions in an exemplary embodiment. Processing system 2000 is operable to perform the above operations by executing programmed instructions tangibly embodied on computer readable storage medium 2012. In this regard, embodiments of the invention can take the form of a computer program accessible via computer-readable medium 2012 providing program code for use by a computer or any other instruction execution system. For the purposes of this description, computer readable storage medium 2012 can be anything that can contain or store the program for use by the computer.

Computer readable storage medium 2012 can be an electronic, magnetic, optical, electromagnetic, infrared, or semiconductor device. Examples of computer readable storage medium 2012 include a solid state memory, a magnetic tape, a removable computer diskette, a random access memory (RAM), a read-only memory (ROM), a rigid magnetic disk, and an optical disk. Current examples of optical disks include compact disk-read only memory (CD-ROM), compact disk-read/write (CD-R/W), and DVD.

Processing system 2000, being suitable for storing and/or executing the program code, includes at least one processor 2002 coupled to program and data memory 2004 through a system bus 2050. Program and data memory 2004 can include local memory employed during actual execution of the program code, bulk storage, and cache memories that provide temporary storage of at least some program code and/or data in order to reduce the number of times the code and/or data are retrieved from bulk storage during execution.

Input/output or I/O devices 2006 (including but not limited to keyboards, displays, pointing devices, etc.) can be coupled either directly or through intervening I/O controllers. Network adapter interfaces 2008 may also be integrated with the system to enable processing system 2000 to become coupled to other data processing systems or storage devices through intervening private or public networks. Modems, cable modems, IBM Channel attachments, SCSI, Fibre Channel, and Ethernet cards are just a few of the currently available types of network or host interface adapters. Display device interface 2010 may be integrated with the system to interface to one or more display devices, such as printing systems and screens for presentation of data generated by processor 2002.

What is claimed is:

1. A system comprising:
a fleet control server that receives a request for medical transport of a patient, and that identifies a fleet of vehicles that provide medical transport;
crew devices at the vehicles that each report a position of a corresponding vehicle; and
a reporting server;
the fleet control server selects a vehicle in the fleet to perform medical transport of the patient based on the position of the vehicle in relation to other vehicles in the fleet, and determines an interested party that is distinct from the patient; and
the reporting server reports selection of the vehicle to the interested party, and transmits vehicle tracking data to the interested party in real time as the vehicle services the request.

2. The system of claim 1 wherein:
the interested party is a caller who has initiated the request for medical transport via an emergency telephone call;
the fleet control server identifies a medical condition of the patient; and
the reporting server transmits bystander instructions to a mobile device of the interested party, based on the medical condition of the patient.

3. The system of claim 1 wherein:
the request indicates one or more types of care to be provided during transport; and
the fleet control server prevents selection of any vehicle that is incapable of providing the types of care.

4. The system of claim 1 wherein:
the fleet control server receives a status of the patient, provided by a crew device of the vehicle in real time while the patient is in the vehicle; and
the reporting server transmits the status of the patient to the interested party.

5. The system of claim 1 wherein:
the fleet control server receives treatment information, indicating medical procedures performed on the patient during medical transport of the patient; and
the reporting server transmits the treatment information to the interested party.

6. The system of claim 1 wherein:
the reporting server transmits an arrival time estimate of the vehicle to the interested party.

7. The system of claim 1 wherein:
there are multiple interested parties; and
the reporting server transmits a pick up arrival time estimate of the vehicle to a first interested party, and transmits a drop off arrival time estimate of the vehicle to a second interested party.

8. The system of claim 1 wherein:
the fleet control server generates a map that includes a location of the interested party, a pick up location for the patient, and a location of the vehicle; and
the reporting server transmits an instruction to present the map at a display of the interested party.

9. The system of claim 1 wherein:
the request indicates a pick up location and a drop off location for the patient.

10. The system of claim 1 wherein:
the request is received from a Computer Aided Dispatch (CAD) system; and
the request includes contact information for the interested party.

11. A method comprising:
receiving a request for medical transport of a patient;
identifying a fleet of vehicles that provide medical transport;
determining a position of each of the vehicles in the fleet as reported by crew devices at the vehicles;
selecting a vehicle in the fleet to perform medical transport of the patient based on the position of the vehicle in relation to other vehicles in the fleet;
determining an interested party that is distinct from the patient;
reporting selection of the vehicle to the interested party; and
transmitting vehicle tracking data to the interested party in real time as the vehicle services the request.

12. The method of claim 11 wherein:
the interested party is a caller who has initiated the request for medical transport via an emergency telephone call; and
the method further comprises:
identifying a medical condition of the patient; and
transmitting bystander instructions to a mobile device of the interested party, based on the medical condition of the patient.

13. The method of claim 11 wherein:
the request indicates one or more types of care to be provided during transport; and
the method further comprises:
preventing selection of any vehicle that is incapable of providing the types of care.

14. The method of claim 11 further comprising:
receiving a status of the patient, provided by a crew device of the vehicle in real time while the patient is in the vehicle; and
transmitting the status of the patient to the interested party.

15. The method of claim 11 further comprising:
receiving treatment information, indicating medical procedures performed on the patient during medical transport of the patient; and
transmitting the treatment information to the interested party.

16. The method of claim 11 further comprising:
transmitting an arrival time estimate of the vehicle to the interested party.

17. The method of claim 11 wherein:
there are multiple interested parties; and
the method further comprises transmitting a pick up arrival time estimate of the vehicle to a first interested party, and transmitting a drop off arrival time estimate of the vehicle to a second interested party.

18. The method of claim 11 further comprising:
generating a map that includes a location of the interested party, a pick up location for the patient, and a location of the vehicle; and
transmitting an instruction to present the map at a display of the interested party.

19. The method of claim 11 wherein:
the request indicates a pick up location and a drop off location for the patient.

20. A non-transitory computer readable medium embodying programmed instructions which, when executed by a processor, are operable for performing a method comprising:
receiving a request for medical transport of a patient;
identifying a fleet of vehicles that provide medical transport;
determining a position of each of the vehicles in the fleet as reported by crew devices at the vehicles;
selecting a vehicle in the fleet to perform medical transport of the patient based on the position of the vehicle in relation to other vehicles in the fleet;
determining an interested party that is distinct from the patient;
reporting selection of the vehicle to the interested party; and
transmitting vehicle tracking data to the interested party in real time as the vehicle services the request.
